(19) United States
(12) Patent Application Publication
(10) Pub. No.: US 2023/0218493 A1
(43) Pub. Date: Jul. 13, 2023

(54) ANTI-AGING FORMULA

(71) Applicant: Sincerus Pharmaceuticals, Inc., Pompano Beach, FL (US)

(72) Inventor: Abul K. BHUIYAN, Davie, FL (US)

(21) Appl. No.: 18/091,578

(22) Filed: Dec. 30, 2022

Related U.S. Application Data
(60) Provisional application No. 63/295,066, filed on Dec. 30, 2021.

(51) Int. Cl. A61K 8/36; A61K 8/49; A61K 8/73; A61K 8/65; A61K 8/9789; A61Q 19/08

(52) U.S. Cl. CPC A61K 8/361; A61K 8/4926; A61K 8/735; A61K 8/65; A61K 8/9789; A61K 8/498; A61Q 19/08; A61K 2800/85

(57) ABSTRACT

The present invention provides improved anti-aging skin cream formulations. The anti-aging cream formulations contain tretinoin, niacinamide, hyaluronic acid, collagen, red wine extract, α-tocopheryl acetate, Butyrospermum parkii (shea butter) extract, and suspendisse cream. The present invention also provides kits containing the formulations, methods of making the formulations as well as methods of using these formulations to visibly reduce signs of aging.

### CROSS REFERENCE TO RELATED APPLICATIONS

This application claims the benefit of U.S. Provisional Application No. 63/295,066, filed Dec. 30, 2021, the entirety of which is incorporated by reference herein.

### TECHNICAL FIELD

This application claims the benefit of U.S. Provisional Application No. 63/295,066, filed Dec. 30, 2021, the disclosure of which is incorporated by reference in its entirety.

This invention relates to improved anti-aging cream formulations that rely on the unique benefits provided by using tretinoin and niacinamide together.

### BACKGROUND

As human gets older, the skin develops wrinkles and other signs of aging including a reduction in the hydro-lipid protective skin barrier.

While some anti-aging cream formulations combine tretinoin and niacinamide to rely on the beneficial effects provided by these compounds, they do not adequately reduce the visible appearance of skin aging.

What is needed is an improved anti-aging cream formulation that reduces the visible appearance of skin aging.

### SUMMARY

The present disclosure provides anti-aging cream formulations containing at least tretinoin; niacinamide; hyaluronic acid; collagen; red wine extract; α-tocopheryl acetate; Butyrospermum parkii (shea butter) extract, and Argania spinosa kernel oil; and suspendisse cream.

In one embodiment, the anti-aging cream formulation contains: from about 0.03 to about 0.07 w/w % of tretinoin; from about 3.5 to about 4.5 w/w % of niacinamide; from about 0.2 to about 0.7 w/w % of hyaluronic acid; from about 0.04 to about 0.065 w/w % of collagen; from about 1.5 to about 2.5 w/w % of red wine extract; from about 0.05 to about 0.4 w/w % of α-tocopheryl acetate; and the remainder in w/w % of suspendisse cream.

In another embodiment, the anti-aging cream formulation of contains: about 0.055 w/w % of tretinoin; about 4.0 w/w % of niacinamide; about 0.5 w/w % of hyaluronic acid; about 0.05 w/w % of collagen; about 2.0 w/w % of red wine extract; about 0.2 w/w % of α-tocopheryl acetate; about 2.0 w/w % of Butyrospermum parkii (shea butter) extract; and the remainder in w/w % of suspendisse cream.

In certain embodiments, the hyaluronic acid is hyaluronic acid sodium salt. In other embodiments, the Butyrospermum parkii (shea butter) extract is preservative-free.

In some embodiments, the collagen is a 1% solution of collagen. In those embodiments, the formulation may contain: about 0.055 w/w % of tretinoin; about 4.0 w/w % of niacinamide; about 0.5 w/w % of hyaluronic acid; about 0.5 w/w % of the 1% collagen solution; about 2.0 w/w % of red wine extract; about 0.2 w/w % of α-tocopheryl acetate; about 2.0 w/w % of Butyrospermum parkii (shea butter) extract; and about 86.245 w/w % of suspendisse cream. Alternatively, the anti-aging cream formulation contains: from about 0.04 to about 0.06 w/w % of tretinoin; from about 3.8 to about 4.2 w/w % of niacinamide; from about 0.3 to about 0.6 w/w % of hyaluronic acid; from about 4.5 to about 6 w/w % of the 1% collagen solution; from about 1.7 to about 2.3 w/w % of red wine extract; from about 0.1 to about 0.3% of α-tocopheryl acetate; from about 1.7 to about 2.3 w/w % of Butyrospermum parkii (shea butter) extract; and from about 85 to about 87 w/w % of suspendisse cream.

The anti-aging cream formulations uniquely provide for improved stability, for example, no degradation in the potency of tretinoin and niacinamide is observed after storage for 1 to 3 months, alternatively a month, alternatively two months, alternatively three months when stored at 20° C. to 25° C./ambient humidity.

The disclosure also provides for kits containing the anti-aging cream formulation whereby the anti-aging cream formulation is packaged in a container. The kits may also include an applicator and/or instructions for use.

The disclosure also provides for methods of visibly reducing signs of skin aging comprising applying the anti-aging cream formulations to the skin of a patient. For example, the anti-aging cream formulation is applied to the face of the patient. The methods reduce the visible appearance of wrinkles when compared to skin to which the anti-aging cream formulation was not applied.

In addition, the disclosure provides for methods of restoring the hydro-lipid protective skin barrier, moisturizing skin, reducing skin inflammation, or reducing skin redness comprising applying the anti-aging cream formulation to the skin of a human, such as e.g., the face.

The disclosure also provides for methods of making an anti-aging cream formulation comprising mixing tretinoin, niacinamide, hyaluronic acid, collagen, red wine extract, α-tocopheryl acetate, Butyrospermum parkii (shea butter) extract, and suspendisse cream. The methods may also include packing the formulation in a container and/or storing the anti-aging cream formulation. The methods may include mixing a 0.1% solution of collagen. The methods generate formulations that do not exhibit any degradation in the potency of tretinoin and niacinamide after storage for 1 to 3 months when stored at 20° C. to 25° C./ambient humidity.

In one embodiment, the methods include providing a sufficient amount of tretinoin, niacinamide, hyaluronic acid, collagen, red wine extract, α-tocopheryl acetate, Butyrospermum parkii (shea butter) extract, and suspendisse cream to generate an anti-aging cream formulation comprising: about 0.055 w/w % of tretinoin; about 4.0 w/w % of niacinamide; about 0.5 w/w % of hyaluronic acid; about 0.5 w/w % of the 1% collagen solution; about 2.0 w/w % of red wine extract; about 0.2 w/w % of α-tocopheryl acetate; about 2.0 w/w % of, Butyrospermum parkii (shea butter) extract; and about 86.245 w/w % of suspendisse cream.

In another embodiment, the methods include providing a sufficient amount of tretinoin, niacinamide, hyaluronic acid, collagen, red wine extract, α-tocopheryl acetate, Butyrospermum parkii (shea butter) extract, and suspendisse cream to generate an anti-aging cream formulation comprising: from about 0.04 to about 0.06 w/w % of tretinoin; from about 3.8 to about 4.2 w/w % of niacinamide; from about 0.3 to about 0.6 w/w % of hyaluronic acid; from about 4.5 to about 6 w/w % of the 1% collagen solution; from about 1.7 to about 2.3 w/w % of red wine extract; from about 0.1 to about 0.3% of α-tocopheryl acetate; from about 1.7 to about 2.3 w/w % of Butyrospermum parkii (shea butter) extract; and from about 85 to about 87 w/w % of suspendisse cream.

Other features and advantages of the invention will be apparent from the detailed description and examples that follow.

### DETAILED DESCRIPTION

The present disclosure provides anti-aging cream formulations that combine the beneficial properties of tretinoin and niacinamide with hyaluronic acid; collagen; red wine extract; α-tocopheryl acetate; Butyrospermum parkii (shea butter) extract; and suspendisse cream. The formulations of the disclosure have great stability with almost no potency loss after storage for 1-3 months.

The anti-aging cream formulation may be used to visibly reduce the appearance of wrinkles. In addition, the disclosure provides methods of making the formulation and kits containing the formulations.

The anti-aging cream formulations provide a variety of benefits, such as improvement of skin appearance, stress reduction, reduction of appearance of lines and wrinkles, visible reduction of facial lines and wrinkles, and/or reduction of skin redness.

The general description and the following detailed description are exemplary and explanatory only and are not restrictive of the invention, as defined in the appended claims. Other aspects of the present invention will be apparent to those skilled in the art in view of the detailed description of the invention as provided herein.

Each of the individual embodiments described and illustrated herein have discrete components and features which may be readily separated from or combined with the features of any of the other several embodiments without departing from the scope or spirit of the present invention. Any recited method may be carried out in the order of events recited or in any other order which is logically possible.

### Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Throughout the description and claims of this specification, the words “comprise” and “contain” and variations of the words, for example “comprising” and “comprises,” mean “including but not limited to,” and are not intended to (and do not) exclude other components.

As used herein, the term “consisting of” excludes any element, step, or ingredient not specified in the claim element.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

Anti-Aging Cream Formulations

One aspect of the disclosure is related to improved anti-aging cream formulations. The anti-aging cream formulations of the disclosure contain tretinoin (all-trans retinoic acid) and niacinamide along with other ingredients.

The anti-aging cream formulations of the disclosure contain:

a) tretinoin;

b) niacinamide;

c) hyaluronic acid;

d) collagen;

e) red wine extract;

f) α-tocopheryl acetate;

g) Butyrospermum parkii (shea butter) extract; and

h) suspendisse cream.

The anti-aging cream formulations of the disclosure associate a complex of fats and oils, which play an essential role in restoring the hydro-lipid protective skin barrier, contributing to a softening, nourishing, and moisturizing of sensitive skin (affected by external factors, dehydration, or wrinkles), with red wine extract having regenerative properties, collagen and α-tocopherol acetate having anti-aging action for the skin.

Tretinoin

The formulations of the disclosure contain tretinoin. Tretinoin can also be referred to as all-trans retinoic acid (ATRA). Tretinoin is known to improve the appearance of surface wrinkles, fine lines, and darks spots. The tretinoin can be prepared in one or more forms including a cream, a gel, a lotion, an ointment, and the like. In one embodiment, the formulation contains about 0.055 w/w % of tretinoin. In another embodiment, the formulation contains from about 0.04 w/w % to about 0.06 w/w % of tretinoin including any and all increments therebetween. In an alternate embodiment, the formulation contains from about 0.03 w/w % to about 0.07 w/w % of tretinoin including any and all increments therebetween. In an alternate embodiment, the formulation contains from about 0.025 w/w % to about 0.075 w/w % of tretinoin, including any and all increments therebetween.

Niacinamide

The formulations of the disclosure also contain niacinamide. Niacinamide can also be referred to as nicotinamide. The niacinamide can be prepared in one or more forms including as a serum, a liquid solution, a cream, a powder, a paste and the like for adding to the formulation of the present disclosure. The formulations may contain about 4.0 w/w %, alternatively from about 3.8 w/w % to about 4.2 w/w %, alternatively from about 3.5 w/w % to about 4.5 w/w % of niacinamide, including any and all increments therebetween.

Hyaluronic Acid

The formulations of the disclosure comprise hyaluronic acid. The hyaluronic acid may also be referred to as hyaluronan. In one embodiment, the hyaluronic acid is present in the form of a hyaluronic acid sodium salt, a hyaluronic acid potassium salt, or a combination thereof. The formulations may contain about 0.5 w/w %, alternatively from about 0.3 w/w % to about 0.6 w/w %, alternatively from about 0.2 w/w % to about 0.7 w/w % of hyaluronic acid (e.g., hyaluronic acid sodium salt), including any and all increments therebetween.

Collagen

The formulation of the disclosure also comprise collagen. The presence of collagen, natural component of the skin, stimulates neo-fibrillogenic and ensures permanent maintenance of skin hydration. The collagen may include type I collagen, type III collagen, type II collagen, or type V collagen including one or one or more combinations thereof. In some embodiments, the collagen includes hydrolyzed collagen. The collagen may be present in amounts of about 0.05 w/w %, alternatively from about 0.045 w/w % to about 0.06 w/w %, alternatively from about 0.04 w/w % to about 0.065 w/w %, including any and all increments therebetween.

In certain embodiments, the formulations of the disclosure comprise a 1% solution of collagen. In these embodiments, the formulation contains about 5.0 w/w %, alternatively, from about 4.5 w/w % to about 6 w/w %, alternatively from about 4 w/w % to about 6.5 w/w % of collagen (1% solution). In other embodiments, a more concentrated solution of collagen is used. For example, in some embodiments, the solution of collage contains from about 5.0 w/w % to about 7.0 w/w % of collagen, from about 5.5 w/w % to about 7.5 w/w % collagen, from about 6.0 w/w % to about 8.0 w/w %, from about 6.5 w/w % to about 8.5 w/w %, from about 7.0 w/w % to about 9.0 w/w %, from about 7.5 w/w % to about 9.5 w/w %, from about 8.0 w/w % to about 10 w/w %, and any and all increments therebetween.

Red Wine Extract

The formulations of the disclosure contain red wine extract. Red wine extract is rich in polyphenols (e.g., resveratrol), sugars, vitamins, and minerals. It stimulates, protects, hydrates, and regenerates the skin.

In one embodiment, the formulation contains about 2.0 w/w % of the red wine extract. In another embodiment, the formulation contains from about 1.7 w/w % to about 2.3 w/w % of the red wine extract. In an alternate embodiment, the formulation contains from about 1.5 w/w % to about 2.5 w/w % of the red wine extract, including any and all increments therebetween.

α-Tocopheryl Acetate

In addition, the formulations of the disclosure contain α-tocopheryl acetate (also known as α-tocopherol acetate or Vitamin E acetate). α-tocopheryl acetate is an effective antioxidant, helping to combat skin aging processes caused by free radicals. It softens the skin and improves skin elasticity. The formulations of the present disclosure may contain about 0.2 w/w % α-tocopheryl acetate. Alternatively, formulations may contain from about 0.1 w/w % to about 0.3 w/w %, alternatively from about 0.05 w/w % to about 0.4 w/w % of α-tocopheryl acetate.

B. parkii Extract

The formulations of the disclosure also contain Butyrospermum parkii (shea butter) extract. In one embodiment, the formulation contains about 2.0 w/w % of the Butyrospermum parkii (shea butter) extract. In another embodiment, the formulation contains from about 1.7 w/w % to about 2.3 w/w % of the Butyrospermum parkii (shea butter) extract. In another embodiment, the formulation contains from about 1.5 w/w % to about 2.5 w/w % of the mixture of Butyrospermum parkii (shea butter) extract.

Preferably the Butyrospermum parkii (shea butter) extract is a preservative-free. Shea butter contains a high concentration of vitamins and fatty acids. Shea butter is known for its excellent softening, moisturizing and nourishing properties. Shea butter also possesses beneficial properties including anti-inflammatory, antioxidant, antibacterial, and antifungal properties. Shea butter is also known to increase collagen production, promote cell regeneration, reduce appearance of skin defects including stretch marks, scars, wrinkles and the like. Shea butters provides sun protection, sooths skin inflammatory conditions including eczema, dermatitis, psoriasis and the like. Shea butter also possesses good spread-ability while providing a non-greasy feeling to the skin.

In certain embodiments, the Butyrospermum parkii (shea butter) extract may be a commercially available mixture.

Suspendisse Cream

The formulations of the disclosure also contain suspendisse cream. Suspendisse is a highly elegant hydrophilic cream base vehicle. It is pleasant and soft, non-greasy skin feel and excellent hydration effect to support skin balance. The suspendisse cream may account for the reminder for the formulation w/w % (based on combining any of the amounts above). In one embodiment, the formulations contain about 86.245 w/w % of suspendisse cream. In another embodiment, the formulations contain from about 85 w/w % to about 87 w/w % of suspendisse cream. In yet another embodiment, the formulations contain from about 84.5 w/w % to about 87.5 w/w % of suspendisse cream. In yet another embodiment, the formulations contain from about 84 w/w % to about 88 w/w % of suspendisse cream. In yet another embodiment, the formulations contain from about 82 w/w % to about 90 w/w % of suspendisse cream, including any and all increments therebetween.

The suspendisse cream contains a mixture of cetearyl alcohol, Cyclopentasiloxane, dimethicone, ethyhexyglycerin, hydoxyethyl acrylate/sodium acryloyldimethyl taurate copolymer, paraffine liquidum, phenoxyethanol, polysorbate 60, sodium hyaluronate, sorbitan isostearate, squalane, and purified water. In certain aspects, suspendisse cream is an excellent emollient, with a good spreadability. Furthermore, suspendisse cream can sooth and nourish the skin without leaving a residual greasy feeling to the recipient.

In certain embodiments, the anti-aging cream formulations of the disclosure contains at least the following components in the any of the amounts specified in any of the embodiments shown in Tables A-D: tretinoin; niacinamide; hyaluronic acid; collagen; red wine extract; α-tocopheryl acetate; Butyrospermum parkii (shea butter) extract; and suspendisse cream.

TABLE A
Examples of formulations according to the disclosure

| | Embodiment | | |
|---|---|---|---|
| | A | B | C |
| Ingredient | w/w % | w/w % | w/w % |
| Tretinoin | about 0.055 | from about 0.04 to | from about 0.03 to |
| | | about 0.06 | about 0.07 |
| Niacinamide | about 4.0 | from about 3.8 to | from about 3.5 to |
| | | about 4.2 | about 4.5 |
| Hyaluronic Acid Sodium Salt or | about 0.5 | from about 0.3 to | from about 0.2 to |
| Hyaluronic Acid | | about 0.6 | about 0.7 |
| Collagen (1% solution) | about 5.0 | from about 4.5 to | from about 4 to |
| | | about 6 | about 6.5 |
| red wine extract | about 2.0 | from about 1.7 to | from about 1.5 to |
| | | about 2.3 | about 2.5 |
| α-tocopheryl acetate | about 0.2 | from about 0.1 to | from about 0.05 to |
| | | about 0.3 | about 0.4 |
| Butyrospermum parkii (shea | about 2.0 | from about 1.7 to | from about 1.5 to |
| butter) extract | | about 2.3 | about 2.5 |
| Suspendisse Cream | about 86.245 | from about 85 to | from about 84.5 to |
| | | about 87 | about 87.5 |

TABLE B
Examples of formulations according to the disclosure

| | Embodiment | | |
|---|---|---|---|
| | A | B | C |
| Ingredient | w/w % | w/w % | w/w % |
| Tretinoin | about 0.055 | from about 0.04 to | from about 0.03 to |
| | | about 0.06 | about 0.07 |
| Niacinamide | about 4.0 | from about 3.8 to | from about 3.5 to |
| | | about 4.2 | about 4.5 |
| Hyaluronic Acid Sodium Salt or | about 0.5 | from about 0.3 to | from about 0.2 to |
| Hyaluronic Acid | | about 0.6 | about 0.7 |
| Collagen (1% solution) | about 5.0 | from about 4.5 to | from about 4 to |
| | | about 6 | about 6.5 |
| red wine extract | about 2.0 | from about 1.7 to | from about 1.5 to |
| | | about 2.3 | about 2.5 |
| α-tocopheryl acetate | about 0.2 | from about 0.1 to | from about 0.05 to |
| | | about 0.3 | about 0.4 |
| Butyrospermum parkii (shea | about 2.0 | from about 1.7 to | from about 1.5 to |
| butter) extract | | about 2.3 | about 2.5 |

| | |
|---|---|
| Suspendisse Cream | remainder |

TABLE C
Examples of formulations according to the disclosure

| | Embodiment | | |
|---|---|---|---|
| | A | B | C |
| Ingredient | w/w % | w/w % | w/w % |
| Tretinoin | about 0.055 | from about 0.04 to | from about 0.03 to |
| | | about 0.06 | about 0.07 |
| Niacinamide | about 4.0 | from about 3.8 to | from about 3.5 to |
| | | about 4.2 | about 4.5 |
| Hyaluronic Acid Sodium Salt or | about 0.5 | from about 0.3 to | from about 0.2 to |
| Hyaluronic Acid | | about 0.6 | about 0.7 |
| Collagen | about 0.05 | from about 0.045 to | from about 0.04 to |
| | | about 0.06 | about 0.065 |
| red wine extract | about 2.0 | from about 1.7 to | from about 1.5 to |
| | | about 2.3 | about 2.5 |
| α-tocopheryl acetate | about 0.2 | from about 0.1 to | from about 0.05 to |
| | | about 0.3 | about 0.4 |
| Butyrospermum parkii (shea | about 2.0 | from about 1.7 to | from about 1.5 to |
| butter) extract | | about 2.3 | about 2.5 |
| Suspendisse Cream | about 86.245 | from about 85 to | from about 84.5 to |
| | | about 87 | about 87.5 |

TABLE D
Examples of formulations according to the disclosure

| | Embodiment | | |
|---|---|---|---|
| | A | B | C |
| Ingredient | w/w % | w/w % | w/w % |
| Tretinoin | about 0.055 | from about 0.04 to | from about 0.03 to |
| | | about 0.06 | about 0.07 |
| Niacinamide | about 4.0 | from about 3.8 to | from about 3.5 to |
| | | about 4.2 | about 4.5 |
| Hyaluronic Acid Sodium Salt or | about 0.5 | from about 0.3 to | from about 0.2 to |
| Hyaluronic Acid | | about 0.6 | about 0.7 |
| Collagen | about 0.05 | from about 0.045 to | from about 0.04 to |
| | | about 0.06 | about 0.065 |
| red wine extract | about 2.0 | from about 1.7 to | from about 1.5 to |
| | | about 2.3 | about 2.5 |
| α-tocopheryl acetate | about 0.2 | from about 0.1 to | from about 0.05 to |
| | | about 0.3 | about 0.4 |
| Butyrospermum parkii (shea | about 2.0 | from about 1.7 to | from about 1.5 to |
| butter) extract | | about 2.3 | about 2.5 |

| | |
|---|---|
| Suspendisse Cream | remainder |

In some embodiments, the anti-aging cream formulations described above may also contain squalane. When squalane is included in the formulations, the amount of α-tocopheryl acetate in the formulations is increased. In certain embodiments, the formulations also contain from about 1.8 to about 2.2 w/w % of squalane. In other embodiments, the formulations contain from about 1.8 to about 2.2 w/w % of squalane and from about 0.2 to about 0.4% of α-tocopheryl acetate.

In one embodiment, the anti-aging cream formulation comprises the composition listed in Table 1-1 below.

The anti-aging cream formulations of the disclosure have improved stability compared to anti-aging cream formulations containing only tretinoin; niacinamide; hyaluronic acid; and suspendisse cream.

In particular, in the anti-aging cream formulations no degradation in the potency of tretinoin and niacinamide is observed after storage for 1 to 3 months, alternatively for 1 month, alternatively for 2 months, alternatively for 3 months, when stored at 20° C. to 25° C. at ambient humidity. Furthermore, the anti-aging formulations have a pH of about 5.0, alternatively a pH ranging from about 4.8 to about 6.0.

Kits

In another embodiment, the invention is directed to kit containing the anti-aging cream formulation as disclosed herein, whereby the anti-aging cream formulation is packaged in a container. The kit may optionally include an applicator and/or instructions for use.

Use of the Anti-Aging Cream Formulations

In another aspect, the present disclosure provides for a variety of uses of the anti-aging cream formulations. For example, the anti-aging cream formulations may be used to restore the hydro-lipid protective skin barrier, moisturizing the skin, reduce skin inflammation, or reduce skin redness.

In another embodiment, the disclosure provides for methods of visibly reducing signs of skin aging. The method comprises applying an anti-aging cream formulation of the disclosure to the skin of a human. In certain embodiments, the methods reduce the visible appearance of wrinkles when compared to skin to which the anti-aging cream formulation was not applied.

Methods of Making the Anti-Aging Cream Formulations

Other embodiments of the invention are directed to methods of making the anti-aging cream formulations of the disclosure. The anti-aging cream formulations of the disclosure are generated by step-wise addition.

In one embodiment, each of tretinoin; niacinamide; hyaluronic acid; collagen; red wine extract; α-tocopheryl acetate; Butyrospermum parkii (shea butter) extract; are added to the suspendisse cream separately. In certain embodiments, some of the components are mixed together prior to adding the cream.

The method may also include the step of packaging the formulation in a container. In addition, the method may include storing the formulation.

In another embodiment, the method includes the following:
calculate the required quantity of each ingredient for total amount to be prepared;
accurately weigh and/measure each ingredient;
transfer tretinoin, niacinamide, hyaluronic acid, Collagen (such as e.g., 1% collagen solution), Red wine extract, a tocopheryl acetate, shea butter, 50% amount of Suspendisse Cream to a mixer and mix for about 15 minutes at a speed of about 1200 rpm;
add the remaining amount of Suspendisse cream to the mixer and mix for about 20 minutes at a speed of about 750 rpm; and
package the final anti-aging formulation.

In certain embodiments, the method also includes the step of one or more sample for homogeneity testing. In certain embodiments, the packaging includes packaging the formulation in an airless pump container. In other embodiments, the method includes packaging the formulation in a container.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1—Anti-Aging Cream

An exemplary anti-aging cream according to the instant disclosure was formulated. The components of the anti-aging cream are shown in Table 1-1 below.

TABLE 1-1
Exemplary Anti-aging cream

| Ingredient | w/w % |
|---|---|
| Tretinoin, USP (10% excess) | 0.055 |
| Niacinamide, USP | 4.0 |
| Hyaluronic Acid Sodium Salt | 0.5 |
| Collagen 1% | 5.0 |
| Red wine extract | 2.0 |
| α-Tocopheryl Acetate | 0.2 |
| Shea butter | 2.0 |
| Suspendisse Cream | 86.245 |

### Example 2—Stability Tests

As tretinoin and niacinamide degrade as a function of time. The stability of the formulation shown in Table 1-1 was investigated. Specifically, the amounts of tretinoin and niacinamide were measured after storage for one month and three months at INT (intermediate conditions at 30° C.±2° C./65% relative humidity (65%±5%) and CRT (20° C. to 25° C./ambient humidity). The results of the stability testing are shown in Tables 2-1 and 2-2 below.

TABLE 2-1
Results of Stability Testing
% Label Claim

| Tretinoin | | | Niacinamide | | |
|---|---|---|---|---|---|
| T = 0 | 1M INT | 1M CRT | T = 0 | 1M INT | 1M CRT |
| 110.1% | 107.0% | 105.4% | 102.5% | 103.1% | 102.9% |

TABLE 2-2
Results of Stability Testing
% Label Claim

| Tretinoin | | | Niacinamide | | |
|---|---|---|---|---|---|
| T = 0 | 3M INT | 3M CRT | T = 0 | 3M INT | 3M CRT |
| 110.1% | 104.9 | 105.4% | 102.5% | 102.4 | 101.8 |

The formulation appears to be very stable. T=0 and one month (1M)

Intermediate and CRT data are almost identical and no degradation in potency was observed.

In addition, the stability of the formulation found in Table 1-1 was tested under various temperature and humidity conditions. The results of those stability tests are found in Table 2-3, Table 2-4, and Table 2-5 below. Stability results were obtained using formulation number 271036 version 01 was assessed. This formulation was identified as 4% Niacinamide/Tretinoin 0.05% Cream compounded 20 Apr. 2022 and assigned Lot Number 271036AECACACC@7. This batch was configured with a 30 mL airless pump. The stability assessment was initiated Apr. 29, 2022.

TABLE 2-3
Controlled Room Temperature Stability (CRT): 25° C. ± 2° C./60% Relative
Humidity (RH) ± 5%

| | | | Time Point (Check One) [symbol] Months □ Days | | | |
|---|---|---|---|---|---|---|
| | | | T = 0 | T = 1 M | T = 3 M | T = 6 M |
| | | | QC22-1595 | QC22-1999 | QC22-2721 | QC22-3793 |
| | | | Pull Date: | | | |
| | | | N/A | 31 May 2022 | 29 Jul. 2022 | 28 Oct. 2022 |
| Test | Method | Specification | Result/Date | Result/Date | Result/Date | Result/Date |
| Color | TM-36 | Yellow Off | Yellow Off White | Yellow Off White | Yellow Off White | Yellow Off White |
| | | White to | 29 Apr. 2022 | 31 May 2022 | 1 Aug. 2022 | 28 Oct. 2022 |
| | | Light Yellow | | | | |
| | | Reference | LN088 p. 42 | LN088 p. 57 | LN088 p. 88 | LN111 p. 38 |
| Appearance | TM-36 | Cream | Cream | Cream | Cream | Cream |
| | | | 29 Apr. 2022 | 31 May 2022 | 1 Aug. 2022 | 28 Oct. 2022 |
| | | Reference | LN088 p. 42 | LN088 p. 57 | LN088 p. 88 | LN111 p. 38 |
| pH | TM-37 | 5.00-8.00 | 5.13 | 5.13 | 5.09 | 5.11 |
| | | | 29 Apr. 2022 | 31 May 2022 | 1 Aug. 2022 | 28 Oct. 2022 |
| | | Reference | LB075 p. 29 | LB095 p. 02 | LB115 p. 01 | LB115 p. 79 |
| Assay | TM-14 | 90.0%-110.0% | 100.9% | 100.4% | 99.4% | 98.6% |
| Niacinamide | | | 2 May 2022 | 7 Jul. 2022 | 2 Aug. 2022 | 1 Nov. 2022 |
| | | Reference | LN094 p. 53 | LN102 p. 65 | LN107 p. 28 | LN114 p. 39 |
| Assay | TM-21 | 90.0%-120.0% | 107.2% | 111.8% | 110.7% | 112.0% |
| Tretinoin | | | 2 May 2022 | 22 Jul. 2022 | 2 Aug. 2022 | 2 Nov. 2022 |
| | | Reference | LN095 p. 60 | LN104 p. 48 | LN105 p. 22 | LN119 p. 16 |
| Dispensing | TM-36 | Report Results | Dispensed | Dispensed | Dispensed | Dispensed |
| Test | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Texture | TM-36 | Report Results | Smooth | Smooth | Smooth | Smooth |
| | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Microscopic | N/A | Report Results | Clear and yellow | N/A | N/A | N/A |
| Exam | | | plates crystals | | | |
| | | | 11 Jul. 2022 | | | |
| | | Reference | LN081 p. 36 | N/A | N/A | N/A |

Results from stability testing performed at room temperature (25° C., 60% RH) are shown in Table 2-3. Values including color, appearance, pH, niacinamide, tretinoin, dispensability, texture, and evaluation under a microscope were assessed.

TABLE 2-4
Intermediate (INT) Stability: 30° C. ± 2° C./65% RH ± 5%

| | | | Time Point (Check One) [symbol] Months □ Days | | | |
|---|---|---|---|---|---|---|
| | | | T = 0 | T = 1 M | T = 3 M | T = 6 M |
| | | | QC22-1595 | QC22-2000 | QC22-2722 | QC22-3794 |
| | | | Pull Date: | | | |
| | | | N/A | 31 May 2022 | 29 Jul. 2022 | 28 Oct. 2022 |
| Test | Method | Specification | Result/Date | Result/Date | Result/Date | Result/Date |
| Color | TM-36 | Yellow Off | Yellow Off White | Yellow Off White | Yellow Off White | Yellow Off White |
| | | White to | 29 Apr. 2022 | 1 Jun. 2022 | 1 Aug. 2022 | 28 Oct. 2022 |
| | | Light Yellow | | | | |
| | | Reference | LN088 p. 42 | LN088 p. 57 | LN088 p. 88 | LN111 p. 38 |
| Appearance | TM-36 | Cream | Cream | Cream | Cream | Cream |
| | | | 29 Apr. 2022 | 1 Jun. 2022 | 1 Aug. 2022 | 28 Oct. 2022 |
| | | Reference | LN088 p. 42 | LN088 p. 57 | LN088 p. 88 | LN111 p. 38 |
| pH | TM-37 | 5.00-8.00 | 5.13 | 4.85 | 5.08 | 5.11 |
| | | | 29 Apr. 2022 | 1 Jun. 2022 | 1 Aug. 2022 | 28 Oct. 2022 |
| | | Reference | LB075 p. 29 | LB095 p. 02 | LB115 p. 01 | LB115 p. 79 |
| Assay | TM-14 | 90.0%-110.0% | 100.9% | 101.0% | 98.7% | 97.5% |
| Niacinamide | | | 2 May 2022 | 7 Jul. 2022 | 2 Aug. 2022 | 1 Nov. 2022 |
| | | Reference | LN094 p. 53 | LN102 p. 65 | LN107 p. 28 | LN120 p. 18 |
| Assay | TM-21 | 90.0%-120.0% | 107.2% | 111.8% | 111.2% | 110.7% |
| Tretinoin | | | 2 May 2022 | 22 Jul. 2022 | 2 Aug. 2022 | 2 Nov. 2022 |
| | | Reference | LN095 p. 60 | LN104 p. 48 | LN105 p. 22 | LN119 p. 16 |
| Dispensing | TM-36 | Report Results | Dispensed | Dispensed | Dispensed | Dispensed |
| Test | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Texture | TM-36 | Report Results | Smooth | Smooth | Smooth | Smooth |
| | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Microscopic | N/A | Report Results | Clear and yellow | N/A | N/A | N/A |
| Exam | | | plates crystals | | | |
| | | | 11 Jul. 2022 | | | |
| | | Reference | LN081 p. 36 | N/A | N/A | N/A |

Results from stability testing performed at room temperature (30° C.±2° C./65% RH±5%) are shown in Table 2-4. Values including color, appearance, pH, niacinamide, tretinoin, dispensability, texture, and evaluation under a microscope were assessed.

TABLE 2-5
Freeze/Thaw Stability Study

| | | | Time Point (Check One) [symbol] Months □ Days | | | |
|---|---|---|---|---|---|---|
| | | | T = 0 | T = 1 M | T = 3 M | T = 6 M |
| | | | QC22-1595 | QC22-2000 | QC22-2722 | QC22-3794 |
| | | | Pull Date: | | | |
| | | | N/A | 31 May 2022 | 29 Jul. 2022 | 28 Oct. 2022 |
| Test | Method | Specification | Result/Date | Result/Date | Result/Date | Result/Date |
| Color | TM-36 | Yellow Off | Yellow Off White | Yellow Off White | Yellow Off White | Yellow Off White |
| | | White to | 29 Apr. 2022 | 1 Jun. 2022 | 1 Aug. 2022 | 28 Oct. 2022 |
| | | Light Yellow | | | | |
| | | Reference | LN088 p. 42 | LN088 p. 57 | LN088 p. 88 | LN111 p. 38 |
| Appearance | TM-36 | Cream | Cream | Cream | Cream | Cream |
| | | | 29 Apr. 2022 | 1 Jun. 2022 | 1 Aug. 2022 | 28 Oct. 2022 |
| | | Reference | LN088 p. 42 | LN088 p. 57 | LN088 p. 88 | LN111 p. 38 |
| pH | TM-37 | 5.00-8.00 | 5.13 | 4.85 | 5.08 | 5.11 |
| | | | 29 Apr. 2022 | 1 Jun. 2022 | 1 Aug. 2022 | 28 Oct. 2022 |
| | | Reference | LB075 p. 29 | LB095 p. 02 | LB115 p. 01 | LB115 p. 79 |
| Assay | TM-14 | 90.0%-110.0% | 100.9% | 101.0% | 98.7% | 97.5% |
| Niacinamide | | | 2 May 2022 | 7 Jul. 2022 | 2 Aug. 2022 | 1 Nov. 2022 |
| | | Reference | LN094 p. 53 | LN102 p. 65 | LN107 p. 28 | LN120 p. 18 |
| Assay | TM-21 | 90.0%-120.0% | 107.2% | 111.8% | 111.2% | 110.7% |
| Tretinoin | | | 2 May 2022 | 22 Jul. 2022 | 2 Aug. 2022 | 2 Nov. 2022 |
| | | Reference | LN095 p. 60 | LN104 p. 48 | LN105 p. 22 | LN119 p. 16 |
| Dispensing | TM-36 | Report Results | Dispensed | Dispensed | Dispensed | Dispensed |
| Test | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Texture | TM-36 | Report Results | Smooth | Smooth | Smooth | Smooth |
| | | | 29 Apr. 2022 | 31 May 2022 | 29 Jul. 2022 | 28 Oct. 2022 |
| | | Reference | LN073 p. 89 | LN100 p. 09 | LN100 p. 29 | LN100 p. 49 |
| Microscopic | N/A | Report Results | Clear and yellow | N/A | N/A | N/A |
| Exam | | | plates crystals | | | |
| | | | 11 Jul. 2022 | | | |
| | | Reference | LN081 p. 36 | N/A | N/A | N/A |

Results from stability testing performed under Freeze/Thaw conditions were assessed and are shown in Table 2-5. Values including color, appearance, pH, niacinamide, tretinoin, dispensability, texture, and evaluation under a microscope were assessed.

The following are a number of nonlimiting EXAMPLES of aspects of the disclosure.

One EXAMPLE includes: an anti—aging cream formulation that includes from about 0.03 w/w % to about 0.07 w/w % of tretinoin. The anti—aging cream formulation in addition includes from about 3.5 w/w % to about 4.5 w/w % of niacinamide. The anti—aging cream formulation moreover includes from about 0.2 w/w % to about 0.7 w/w % of hyaluronic acid. The anti—aging cream formulation also includes from about 0.04 w/w % to about 0.065 w/w % of collagen. The anti—aging cream formulation further includes from about 5 w/w % to about 2.5 w/w % of red wine extract. The anti—aging cream formulation in addition includes from about 0.05 w/w % to about 0.4 w/w % of α-tocopheryl acetate. The anti—aging cream formulation moreover includes a remainder in w/w % of suspendisse cream.

The above-noted EXAMPLE may further include any one or a combination of more than one of the following EXAMPLES: The anti—aging cream formulation of the above-noted EXAMPLE where the anti-aging cream formulation may include about 0.055 w/w % of tretinoin; about 4.0 w/w % of niacinamide; about 0.5 w/w % of hyaluronic acid; about 0.05 w/w % of collagen; about 0 w/w % of red wine extract; about 0.2 w/w % of α-tocopheryl acetate; about 0 w/w % of Butyrospermum parkii (shea butter) extract; and the remainder in w/w % of suspendisse cream. The anti—aging cream formulation of the above-noted EXAMPLE where the Butyrospermum parkii (shea butter) extract is preservative-free. The anti—aging cream formulation of the above-noted EXAMPLE where the hyaluronic acid is hyaluronic acid sodium salt. The anti—aging cream formulation of the above-noted EXAMPLE where the collagen may include a 1% solution of collagen. The anti—aging cream formulation of the above-noted EXAMPLE where the anti-aging cream formulation may include: about 0.055 w/w % of tretinoin; about 4.0 w/w % of niacinamide; about 0.5 w/w % of hyaluronic acid; about 0.5 w/w % of the 1% collagen solution; about 2.0 w/w % of red wine extract; about 0.2 w/w % of α-tocopheryl acetate; about 2.0 w/w % of Butyrospermum parkii (shea butter) extract; and about 8245 w/w % of suspendisse cream. The anti—aging cream formulation of the above-noted EXAMPLE where the anti-aging cream formulation may include: from about 0.04 w/w % to about 0.06 w/w % of tretinoin; from about 3.8 w/w % to about 4.2 w/w % of niacinamide; from about 0.3 w/w % to about 0.6 w/w % of hyaluronic acid; from about 4.5 w/w % to about 6 w/w % of the 1% collagen solution; from about 1.7 w/w % to about 2.3 w/w % of red wine extract; from about 0.1% to about 0.3% of α-tocopheryl acetate; from about 1.7 w/w % to about 2.3 w/w % of Butyrospermum parkii (shea butter) extract; and from about 85 w/w % to about 87 w/w % of suspendisse cream. The anti—aging cream formulation of the above-noted EXAMPLE where no degradation in a potency of tretinoin and niacinamide is observed after storage for 1 to 3 months when stored at 20° C. to 25° C./ambient humidity. The anti—aging cream formulation of the above-noted EXAMPLE where no degradation in a potency of tretinoin and niacinamide is observed after storage for a month when stored at 20° C. to 25° C./ambient humidity. The anti—aging cream formulation of the above-noted EXAMPLE where no degradation in a potency of tretinoin and niacinamide is observed after storage for 2 months when stored at 20° C. to 25° C./ambient humidity. The anti—aging cream formulation of the above-noted EXAMPLE where no degradation in a potency of tretinoin and niacinamide is observed after storage for 3 months when stored at 20° C. to 25° C./ambient humidity. The anti—aging cream formulation of the above-noted EXAMPLE where no degradation in a potency of tretinoin and niacinamide is observed after storage for 1 to 3 months when stored at 30° C.±2° C./65%±5% relative humidity. The anti—aging cream formulation of the above-noted EXAMPLE where no degradation in a potency of tretinoin and niacinamide is observed after storage for a month when stored at 30° C.±2° C./65%±5% relative humidity. The anti—aging cream formulation of the above-noted EXAMPLE where no degradation in a potency of tretinoin and niacinamide is observed after storage for 2 months when stored at 30° C.±2° C./65%±5% relative humidity. The anti—aging cream formulation of the above-noted EXAMPLE where no degradation in a potency of tretinoin and niacinamide is observed after storage for 3 months when stored at 30° C.±2° C./65%±5% relative humidity. The anti—aging cream formulation of the above-noted EXAMPLE where the anti-aging cream formulation has a pH of about 5. The anti—aging cream formulation of the above-noted EXAMPLE where the anti-aging cream formulation has a pH ranging from about 4.8 to about 6.0. The anti—aging cream formulation of the above-noted EXAMPLE where the suspendisse cream may include a mixture of cetearyl alcohol, Cyclopentasiloxane, dimethicone, ethyhexyglycerin, hydoxyethyl acrylate/sodium acryloyldimethyl taurate copolymer, paraffine liquidum, phenoxyethanol, polysorbate 60, sodium hyaluronate, sorbitan isostearate, squalane, and purified water. The kit of the above-noted EXAMPLE where the anti-aging cream formulation is packaged in a container. The kit of the above-noted EXAMPLE where the anti-aging cream formulation further may include an applicator and/or instructions for use. The method of the above-noted EXAMPLE to the skin of a human. The method of the above-noted EXAMPLE where the method reduces a visible appearance of wrinkles when compared to skin to which the anti-aging cream formulation was not applied. The method of the above-noted EXAMPLE to the skin of a human. The method of the above-noted EXAMPLE where the method restores a hydro-lipid protective skin barrier. The method of the above-noted EXAMPLE where the method moisturizes the skin. The method of the above-noted EXAMPLE where the method reduces skin inflammation. The method of the above-noted EXAMPLE where the method reduces skin redness. The method of the above-noted EXAMPLE where the anti-aging cream formulation is applied to the face of the human.

One EXAMPLE includes: a method of making an anti-aging cream formulation that includes mixing tretinoin, niacinamide, hyaluronic acid, collagen, red wine extract, α-tocopheryl acetate, Butyrospermum parkii (shea butter) extract, and suspendisse cream.

The above-noted EXAMPLE may further include any one or a combination of more than one of the following EXAMPLES: The method of the above-noted EXAMPLE where the method further may include packaging the anti-aging cream formulation in a container. The method of the above-noted EXAMPLE where the method may include mixing a 0.1% solution of collagen. The method of the above-noted EXAMPLE where the method further includes providing a sufficient amount of tretinoin, niacinamide, hyaluronic acid, collagen, red wine extract, α-tocopheryl acetate, Butyrospermum parkii (shea butter) extract, and suspendisse cream to generate an anti-aging cream formulation having: about 0.055 w/w % of tretinoin; about 4.0 w/w % of niacinamide; about 0.5 w/w % of hyaluronic acid; about 0.5 w/w % of a 1% collagen solution; about 2.0 w/w % of red wine extract; about 0.2 w/w % of α-tocopheryl acetate; about 2.0 w/w % of Butyrospermum parkii (shea butter) extract; and about 86.245 w/w % of suspendisse cream. The method of the above-noted EXAMPLE where the method further includes providing a sufficient amount of tretinoin, niacinamide, hyaluronic acid, collagen, red wine extract, α-tocopheryl acetate, Butyrospermum parkii (shea butter) extract and suspendisse cream to generate an anti-aging cream formulation having: from about 0.04 w/w % to about 0.06 w/w % of tretinoin; from about 3.8 w/w % to about 4.2 w/w % of niacinamide; from about 0.3 w/w % to about 0.6 w/w % of hyaluronic acid; from about 4.5 to about 6 w/w % of a 1% collagen solution; from about 1.7 w/w % to about 2.3 w/w % of red wine extract; from about 0.1% to about 0.3% of α-tocopheryl acetate; from about 1.7 w/w % to about 2.3 w/w % of Butyrospermum parkii (shea butter) extract; and from about 85 w/w % to about 87 w/w % of suspendisse cream. The method of the above-noted EXAMPLE may include storing the anti-aging cream formulation. The method of the above-noted EXAMPLE where the anti-aging cream formulation does not exhibit any degradation in a potency of tretinoin and niacinamide after storage for 1 to 3 months when stored at 20° C. to 25° C./ambient humidity. The method of the above-noted EXAMPLE where the suspendisse cream may include a mixture of cetearyl alcohol, Cyclopentasiloxane, dimethicone, ethyhexyglycerin, hydoxyethyl acrylate/sodium acryloyldimethyl taurate copolymer, paraffine liquidum, phenoxyethanol, polysorbate 60, sodium hyaluronate, sorbitan isostearate, squalane, and purified water. While the invention has been described and illustrated herein by references to various specific materials, procedures, and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

What is claimed is:

1. An anti-aging cream formulation comprising:
from about 0.03 to about 0.07 w/w % of tretinoin;
from about 3.5 to about 4.5 w/w % of niacinamide;
from about 0.2 to about 0.7 w/w % of hyaluronic acid;
from about 0.04 to about 0.065 w/w % of collagen;
from about 1.5 to about 2.5 w/w % of red wine extract;
from about 0.05 to about 0.4 w/w % of α-tocopheryl acetate; and
a remainder in w/w % of suspendisse cream.

2. The anti-aging cream formulation of claim 1, wherein the anti-aging cream formulation comprises
about 0.055 w/w % of tretinoin;
about 4.0 w/w % of niacinamide;
about 0.5 w/w % of hyaluronic acid;
about 0.05 w/w % of collagen;
about 2.0 w/w % of red wine extract;
about 0.2 w/w % of α-tocopheryl acetate;
about 2.0 w/w % of Butyrospermum parkii (shea butter) extract; and
the remainder in w/w % of suspendisse cream.

3. The anti-aging cream formulation of claim 1, wherein the hyaluronic acid is hyaluronic acid sodium salt.

4. The anti-aging cream formulation of claim 2, wherein the Butyrospermum parkii (shea butter) extract is preservative-free.

5. The anti-aging cream formulation of claim 1, wherein the collagen comprises a 1% solution of collagen.

6. The anti-aging cream formulation of claim 5, wherein the anti-aging cream formulation comprises:
about 0.055 w/w % of tretinoin;
about 4.0 w/w % of niacinamide;
about 0.5 w/w % of hyaluronic acid;
about 0.5 w/w % of the 1% collagen solution;
about 2.0 w/w % of red wine extract;
about 0.2 w/w % of α-tocopheryl acetate;
about 2.0 w/w % of Butyrospermum parkii (shea butter) extract; and
about 86.245 w/w % of suspendisse cream.

7. The anti-aging cream formulation of claim 5, wherein the anti-aging cream formulation comprises:
from about 0.04 to about 0.06 w/w % of tretinoin;
from about 3.8 to about 4.2 w/w % of niacinamide;
from about 0.3 to about 0.6 w/w % of hyaluronic acid;
from about 4.5 to about 6 w/w % of the 1% collagen solution;
from about 1.7 to about 2.3 w/w % of red wine extract;
from about 0.1 to about 0.3% of α-tocopheryl acetate;
from about 1.7 to about 2.3 w/w % of Butyrospermum parkii (shea butter) extract; and
from about 85 to about 87 w/w % of suspendisse cream.

8. The anti-aging cream formulation of claim 1, wherein no degradation in a potency of tretinoin and niacinamide is observed after storage for 1 to 3 months when stored at 20° C. to 25° C./ambient humidity.

9. The anti-aging cream formulation of claim 1, wherein no degradation in a potency of tretinoin and niacinamide is observed after storage for a month when stored at 20° C. to 25° C./ambient humidity.

10. The anti-aging cream formulation of claim 1, wherein no degradation in a potency of tretinoin and niacinamide is observed after storage for 2 months when stored at 20° C. to 25° C./ambient humidity.

11. The anti-aging cream formulation of claim 1, wherein no degradation in a potency of tretinoin and niacinamide is observed after storage for 3 months when stored at 20° C. to 25° C./ambient humidity.

12. The anti-aging cream formulation of claim 1, wherein no degradation in a potency of tretinoin and niacinamide is observed after storage for 1 to 3 months when stored at 30° C.±2° C./65%±5% relative humidity.

13. The anti-aging cream formulation of claim 1, wherein no degradation in a potency of tretinoin and niacinamide is observed after storage for a month when stored at 30° C.±2° C./65%±5% relative humidity.

14. The anti-aging cream formulation of claim 1, wherein no degradation in a potency of tretinoin and niacinamide is observed after storage for 2 months when stored at 30° C.±2° C./65%±5% relative humidity.

15. The anti-aging cream formulation of claim 1, wherein no degradation in a potency of tretinoin and niacinamide is observed after storage for 3 months when stored at 30° C.±2° C./65%±5% relative humidity.

16. The anti-aging cream formulation of claim 1, wherein the anti-aging cream formulation has a pH of about 5.

17. The anti-aging cream formulation of claim 1, wherein the anti-aging cream formulation has a pH ranging from about 4.8 to about 6.0.

18. The anti-aging cream formulation of claim 1, wherein the suspendisse cream comprises a mixture of cetearyl alcohol, Cyclopentasiloxane, dimethicone, ethyhexyglycerin, hydoxyethyl acrylate/sodium acryloyldimethyl taurate copolymer, paraffine liquidum, phenoxyethanol, polysorbate 60, sodium hyaluronate, sorbitan isostearate, squalane, and purified water.

19. A kit comprising the anti-aging cream formulation of claim 1, wherein the anti-aging cream formulation is packaged in a container.

20. The kit of claim 19, wherein the anti-aging cream formulation further comprises an applicator and/or instructions for use.

21. A method of visibly reducing signs of skin aging comprising applying the anti-aging cream formulation of claim 1 to the skin of a human.

22. The method of claim 21, wherein the method reduces a visible appearance of wrinkles when compared to skin to which the anti-aging cream formulation was not applied.

23. A method of restoring a hydro-lipid protective skin barrier, moisturizing skin, reducing skin inflammation, or reducing skin redness comprising applying the anti-aging cream formulation of claim 1 to the skin of a human.

24. The method of claim 23, wherein the method restores a hydro-lipid protective skin barrier.

25. The method of claim 23, wherein the method moisturizes the skin.

26. The method of claim 23, wherein the method reduces skin inflammation.

27. The method of claim 23, wherein the method reduces skin redness.

28. The method of claim 23, wherein the anti-aging cream formulation is applied to the face of the human.

29. A method of making an anti-aging cream formulation comprising mixing tretinoin, niacinamide, hyaluronic acid, collagen, red wine extract, α-tocopheryl acetate, Butyrospermum parkii (shea butter) extract, and suspendisse cream.

30. The method of claim 29, wherein the method further comprises packaging the anti-aging cream formulation in a container.

31. The method of claim 29, wherein the method comprises mixing a 0.1% solution of collagen.

32. The method of claim 29, wherein the method further includes providing a sufficient amount of tretinoin, niacinamide, hyaluronic acid, collagen, red wine extract, α-tocopheryl acetate, Butyrospermum parkii (shea butter) extract, and suspendisse cream to generate an anti-aging cream formulation comprising:
about 0.055 w/w % of tretinoin;
about 4.0 w/w % of niacinamide;
about 0.5 w/w % of hyaluronic acid;
about 0.5 w/w % of a 1% collagen solution;
about 2.0 w/w % of red wine extract;
about 0.2 w/w % of α-tocopheryl acetate;
about 2.0 w/w % of Butyrospermum parkii (shea butter) extract; and
about 86.245 w/w % of suspendisse cream.

33. The method of claim 29, wherein the method further includes providing a sufficient amount of tretinoin, niacinamide, hyaluronic acid, collagen, red wine extract, α-tocopheryl acetate, Butyrospermum parkii (shea butter) extract and suspendisse cream to generate an anti-aging cream formulation comprising:
from about 0.04 to about 0.06 w/w % of tretinoin;
from about 3.8 to about 4.2 w/w % of niacinamide;
from about 0.3 to about 0.6 w/w % of hyaluronic acid;
from about 4.5 to about 6 w/w % of a 1% collagen solution;
from about 1.7 to about 2.3 w/w % of red wine extract;
from about 0.1 to about 0.3% of α-tocopheryl acetate;
from about 1.7 to about 2.3 w/w % of Butyrospermum parkii (shea butter) extract; and
from about 85 to about 87 w/w % of suspendisse cream.

34. The method of claim 29, further comprising storing the anti-aging cream formulation.

35. The method of claim 29, wherein the anti-aging cream formulation does not exhibit any degradation in a potency of tretinoin and niacinamide after storage for 1 to 3 months when stored at 20° C. to 25° C./ambient humidity.

36. The method of claim 29, wherein the suspendisse cream comprises a mixture of cetearyl alcohol, Cyclopentasiloxane, dimethicone, ethyhexyglycerin, hydoxyethyl acrylate/sodium acryloyldimethyl taurate copolymer, paraffine liquidum, phenoxyethanol, polysorbate 60, sodium hyaluronate, sorbitan isostearate, squalane, and purified water.
